(19) United States
(12) Patent Application Publication
(10) Pub. No.: US 2016/0169909 A1
(43) Pub. Date: Jun. 16, 2016

(54) COMPOSITION CONTAINING PYRUVATE DEHYDROGENASE KINASE INHIBITOR FOR TREATING CHRONIC INFLAMMATORY PAIN

(71) Applicants: KYUNGPOOK NATIONAL UNIVERSITY INDUSTRY-ACADEMIC COOPERATION FOUNDATION, Daegu (KR); KYUNGPOOK NATIONAL UNIVERSITY HOSPITAL, Daegu (KR)

(72) Inventors: Kyoung Ho SUK, Daegu (KR); In Kyu LEE, Daegu (KR); Mithilesh Kumar JHA, Daegu (KR)

(21) Appl. No.: 14/854,573

(22) Filed: Sep. 15, 2015

(30) Foreign Application Priority Data
Sep. 15, 2014 (KR) 10-2014-0122228

(51) Int. Cl. G01N 33/68; C12Q 1/68; A61K 31/19

(52) U.S. Cl. CPC G01N 33/6893; A61K 31/19; C12Q 1/6883; C12Q 2600/158; G01N 2800/2842; C12Q 2600/136; G01N 2500/00

(57) ABSTRACT

The present invention relates to a composition for treating chronic inflammatory pain containing a pyruvate dehydrogenase kinase (PDK) inhibitor as an active ingredient and a composition for diagnosing chronic inflammatory pain containing a formulation measuring an expression level of mRNA of a PDK gene or a PDK protein. The PDK inhibitor of the present invention reduces an inflammatory response in an animal model where chronic inflammatory pain is induced and diminishes hindpaw edema and pain behaviors. Thus, the PDK inhibitor of the present invention can be useful for treating chronic inflammatory pain. Also, the PDK inhibitor of the present invention can be used for diagnosing chronic inflammatory pain by measuring the PDK expression level, since the PDK expression increases when chronic inflammatory pain is induced.

### CROSS-REFERENCE TO RELATED APPLICATIONS

This application claims the benefit of priority under 35 U.S.C. §119 of Korean Patent Application No. 10-2014-0122228, filed Sep. 15, 2014, which is hereby incorporated by reference in its entirety.

### TECHNICAL FIELD

The present invention relates to compositions for treating chronic inflammatory pain containing pyruvate dehydrogenase kinase (PDK) inhibitors as active ingredients and compositions for diagnosing chronic inflammatory pain containing formulations measuring expression levels of mRNA of PDK genes or PDK proteins.

### BACKGROUND ART

Pain is defined as the most immediate and strongest biological response to noxious stimulus. However, in practical clinics, acute, chronic pain caused by various diseases is the first priority in treatment. The pathological mechanism of pain has not been identified completely, and pain treatment depends on conservative treatment alleviating symptoms, rather than treatment of causes of pain in patients.

Pain is generally classified as acute or chronic pain, and is considered as a complex regulation mechanism through the peripheral nervous system made by primary afferent neurons in spinal ganglia and the central nervous system including spinal cord and brain. Although there may be a debate, it is known that pain is closely associated with a change of various neuroactive substances, particularly excitatory amino acids such as glutamate and inhibitory amino acids such as GABA. DRG consisting of pseudounipolar neurons includes smaller neurons having a diameter of 15 to 30 μm and larger neurons having a diameter up to 100 μm. Neurons are broadly classified into two parts: the peripheral region, cortical zone where a great number of groups of ganglion cell bodies and less nerve fibers are distributed, and the central region, medullary zone where much more nerve fibers and a small number of ganglion cells are distributed. DRG contains various neuroactive substances, such as nitric oxide or substance P, associated with pain regulation.

Recently, studies have vigorously developed in order to identify the mechanism of pain, in relation to development, maintenance, and recovery of pain, for a variety of pain model animals.

Meanwhile, pyruvate dehydrogenase kinase (PDK) is one of proteins regulating the activity of pyruvate dehydrogenase complex (PDC), which is an enzyme involved in glucose oxidation. PDK is known as acting to inactivate PDC for a long term where the activity increases under famines and diabetes, aside from regulation for a short term of inhibiting the activity by pyruvate produced during the time period, which is increased due to acetyl-CoA(acetyl-CoA) and NADH produced during oxidation of fatty acid. However, the role of PDK on inflammatory pain has not been identified yet, and no studies have been made therefor.

The present inventors found that inhibition of PDK leads to excellent effect of alleviating chronic inflammatory pain, during the studies for developing drugs capable of alleviating chronic inflammatory pain, and completed the present invention.

### SUMMARY

The present invention is to provide a composition for treating chronic inflammatory pain containing a PDK inhibitor as an active ingredient.

Also, the present invention is to provide a composition for diagnosing chronic inflammatory pain containing a formulation measuring an expression level of mRNA of a PDK gene or a PDK protein, a kit for diagnosing chronic inflammatory pain containing the composition, and a method for providing information for diagnosing chronic inflammatory pain.

Also, the present invention is to provide a method for screening an agent for treating chronic inflammatory pain using measurement of an expression level of mRNA of a PDK gene or a PDK protein.

A PDK inhibitor of the present invention can reduce an inflammatory response in an animal model where chronic inflammatory pain is induced and reduce hindpaw edema and pain behaviors. Thus, the PDK inhibitor of the present invention can be useful in treatment of chronic inflammatory pain. Also, the PDK inhibitor of the present invention can be used for diagnosing chronic inflammatory pain by measuring PDK expression level, considering that the PDK expression increases when chronic inflammatory pain is induced.

### BRIEF DESCRIPTION OF THE DRAWINGS

FIG. 1 is a view illustrating an mRNA expression pattern of PDK measured through RT-PCR in an animal model where chronic inflammatory pain is induced by CFA.

FIG. 2 is a view illustrating patterns of PDH phosphorylation and PDK protein expression measured through Western blotting in an animal model where chronic inflammatory pain is induced by CFA.

FIG. 3 is a view illustrating PDH phosphorylation measured through immunostaining in an animal model where chronic inflammatory pain is induced by CFA.

FIG. 4 is a view illustrating hindpaw edema (FIG. 4A) and histopathological characteristics (FIG. 4B) in an animal model where chronic inflammatory pain is induced by CFA.

FIG. 5 is a view illustrating thermal paw withdrawal latencies (PWL) for thermal hyperalgesia and paw withdrawal threshold (PWT) for mechanical allodynia in an animal model where chronic inflammatory pain is induced by CFA.

FIG. 6 is a view illustrating a result of measuring infiltration of neutrophils, macrophages and iNOS-positive immune cells in an animal model where chronic inflammatory pain is induced by CFA.

FIG. 7 is a view illustrating a result of measuring expression of TNF-α, IL-1β and IL-6, and lactate level in an animal model where chronic inflammatory pain is induced by CFA.

FIG. 8 is a view illustrating a result of measuring hindpaw edema, thermal paw withdrawal latencies (PWL) for thermal hyperalgesia, and paw withdrawal threshold (PWT) for mechanical allodynia after peripherally administering dichloroacetate in an animal model where chronic inflammatory pain is induced by CFA.

FIG. 9 is a view illustrating a result of measuring hindpaw edema, thermal paw withdrawal latencies (PWL) for thermal hyperalgesia, and paw withdrawal threshold (PWT) for mechanical allodynia after centrally administering dichloroacetate in an animal model where chronic inflammatory pain is induced by CFA.

### DETAILED DESCRIPTION

The present invention provides a composition for treating chronic inflammatory pain containing a pyruvate dehydrogenase kinase (PDK) inhibitor as an active ingredient.

The composition includes a pharmaceutical composition and a food composition.

Hereinafter, the present invention is described in detail.

The PDK of the present invention, pyruvate dehydrogenase kinase, is preferably PDK-2 or PDK-4.

A PDK inhibitor of the present invention may be a PDK activity inhibitor or a PDK expression inhibitor.

The PDK activity inhibitor may be a compound, a peptide, a peptidomimetic, a substrate analog, an aptamer, or an antibody, which bind specifically to a PDK protein, but is not limited thereto.

The compound includes any compounds which bind specifically to a PDK protein and inhibit its activity, and may be, but not limited to, dichloroacetate (DCA), provided in an embodiment of the present invention.

The PDK expression inhibitor may be an antisense nucleotide, RNAi, siRNA, shRNA, or a ribozyme, which binds complementarily to mRNA of a PDK gene, but is not limited thereto.

As used herein, the term “expression inhibition” includes inhibition of gene transcription and inhibition of protein translation. Also, the term includes a complete blockade of gene expression and reduction of expression.

Also, the PDK inhibitor of the present invention may be a pyruvate dehydrogenase complex (PDC) activator activating PDC, since PDK inactivates PDC due to phosphorylation.

The effect of alleviating chronic inflammatory pain may be confirmed in a model where complete Freund's adjuvant (CFA) is administered to induce chronic inflammatory pain.

The composition of the present invention may further contain at least one type of known active ingredients having an effect of alleviating chronic inflammatory pain, with a PDK inhibitor.

The composition of the present invention may further contain a suitable carrier, an excipient, and a diluent, which are commonly used in the manufacture of pharmaceutical compositions. Also, the composition of the present invention may be formulated in the form of oral administration, such as a powder, a granule, a tablet, a capsule, suspension, emulsion, syrup, or an aerosol, an external preparation, a suppository, and a sterile solution for injection, according to a general method. As suitable formulations known in the art, those disclosed in a reference (Remington's Pharmaceutical Science, recently, Mack Publishing Company, Easton Pa.) are preferably used. A carrier, an excipient, and a diluent that may be lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, a mineral oil, and the like. When formulating the composition, the formulation is prepared using a diluent or an excipient, such as a filler, an extender, a binder, a wetting agent, a disintegrant, a surfactant, and the like, which are generally used. A solid formulation for oral administration includes a tablet, a pill, a powder, a granule, a capsule, and the like, and the solid formulation may be prepared by adding at least one of an excipient, e.g., starch, calcium carbonate, sucrose, lactose, gelatin, and the like, to the composition. Also, a lubricant, such as magnesium stearate or talc may be used in addition to a simple excipient. A liquid formulation for oral administration may be suspension, solution, emulsion, syrup, and the like, and various excipients, e.g., a wetting agent, a sweetener, a flavoring agent, a preservative, and the like, may be included in addition to a commonly used diluent, water and liquid paraffin. Examples of formulations for parenteral administration may include a sterile aqueous solution, a non-aqueous solvent, suspension, emulsion, a lyophilized preparation, and a suppository. Examples of the non-aqueous solvent and suspension may include propylene glycol, polyethylene glycol, a vegetable oil such as olive oil, an injectable ester such as ethyl oleate, and the like. Examples of substrates to be used for a suppository may include witepsol, microgol, tween 61, cacao butter, laurinum, glycerogelatin, and the like.

As used herein, the term “administration” means providing the composition of the present invention to a subject according to any proper method.

A preferable amount of the pharmaceutical composition of the present invention to be administered may vary depending on the physical state and body weight of a subject, severity of a disease, type of drug, and routes and duration of administration, but may be properly selected by a person skilled in the art. The PDK inhibitor of the present invention may be administered in an amount of 1 mg/kg to 10000 mg/kg daily. The administration may be performed in a single dose per day or in multiple doses per day.

The pharmaceutical composition of the present invention may be administered to a subject via various routes. All possible administration routes may be expected, e.g., oral administration, rectal or intravenous injection, intramuscular injection, subcutaneous injection, endometrial injection or intracerebroventricular injection.

The composition of the present invention may be used alone or in combination with surgery, radiotherapy, hormone therapy, chemotherapy, and methods using a biological response regulator.

The PDK inhibitor of the present invention may be added to foods or beverages for the purpose of improving chronic inflammatory pain. The foods include various foods, beverages, gums, teas, vitamin complexes, functional foods, and the like, and may include, but are not limited to, specific nutrient foods (e.g., milk formulas, baby, infant foods, and the like), processed meat products, dietary supplements, seasoning foods (e.g., soy sauce, soybean paste, red pepper paste, mixed soy paste, and the like), sauces, confectionaries (i.e., snacks), candies, chocolates, gums, ice creams, milk products (e.g., fermented milk, cheese, and the like), other processed foods, beverages (e.g., fruit beverages, vegetable drinks, fermented drinks, and the like), and natural seasoning. The foods, beverages, or food additives may be prepared according to a general method.

When the PDK inhibitor of the present invention is used as a food composition, the PDK inhibitor may be added as itself, used in combination with other foods or food components, and used properly according to a general method. The amount of active ingredients to be mixed may be suitably determined depending on the purpose of use (for prevention, health or therapeutic treatment). Generally, when manufacturing foods or beverages, the PDK inhibitor of the present invention may be added in an amount of 15 wt % or less, and preferably 10 wt % or less, with respect a raw material. However, in the case of a long-term intake for health, sanitation or diet, the amount may be in the above range or less, and the active ingredients may be used in an amount of the above range or greater, since there is no problem in terms of safety.

The food composition of the present invention may additionally contain a flavoring agent or natural carbohydrate, like general beverages. The natural carbohydrate may be monosaccharide, such as glucose and fructose, disaccharide, such as maltose and sucrose, natural sweetener, such as dextrin and cyclodextrin, or synthetic sweetener, such as saccharin and aspartame. The content of the natural carbohydrate may be generally about 0.01 to 10 g, and preferably about 0.01 to 0.1 g, per 100 ml of the composition of the present invention.

In addition to the above, the composition of the present invention may contain a variety of nutrients, a vitamin, an electrolyte, a flavoring agent, a coloring agent, pectic acid and salts thereof, alginic acid and salts thereof, an organic acid, a protective colloid thickening agent, a pH adjusting agent, a stabilizer, a preservative, glycerin, an alcohol, a carbonating agent used for a carbonated beverage, and the like. Additionally, the composition of the present invention may contain fruit flesh for the preparation of natural fruit juices, fruit juice beverages, and vegetable beverages. These ingredients may be used alone or in combination. The content of the additive is not significantly important, but may be generally selected in a range of 0.01 to 0.1 parts by weight per 100 parts by weight of the composition of the present invention.

In an embodiment, the present invention provides a method for treating chronic inflammatory pain, including administering a PDK inhibitor to a subject.

The PDK of the present invention, pyruvate dehydrogenase kinase, is preferably PDK-2 or PDK-4.

The PDK inhibitor of the present invention may be a PDK activity inhibitor or a PDK expression inhibitor.

The PDK activity inhibitor may be a compound, a peptide, a peptidomimetic, a substrate analog, an aptamer, or an antibody, which binds specifically to a PDK protein, but is not limited thereto.

The PDK expression inhibitor may be an antisense nucleotide, RNAi, siRNA, shRNA, or a ribozyme, which binds complementarily to mRNA of a PDK gene, but is not limited thereto.

Also, the PDK inhibitor of the present invention may be a PDC activator activating PDC, since PDK inactivates PDC due to phosphorylation.

The effect of alleviating chronic inflammatory pain may be confirmed in a model where complete Freund's adjuvant (CFA) is administered to induce chronic inflammatory pain.

In an embodiment, the present invention provides a composition for diagnosing chronic inflammatory pain containing a formulation measuring an expression level of mRNA of a PDK gene or a PDK protein.

As used herein, the term “diagnosis” refers to the determination of pathological conditions. For the purpose of the present invention, the diagnosis means determining the onset of chronic inflammatory pain.

A formulation measuring an mRNA expression level of the PDK gene may include an oligonucleotide, a primer, or a probe, which binds complementarily to mRNA of the PDK gene.

As used herein, the term “primer” refers to a single stranded oligonucleotide which can serve as a starting point for template-directed DNA synthesis under appropriate conditions in an appropriate buffer solution (e.g., four different nucleoside triphosphate and polymers, such as DNA, RNA polymerase or reverse transcriptase) and at an appropriate temperature. The appropriate length of the primer may vary depending on the purpose of use, but may be generally 15 to 30 nucleotides. Generally, a shorter primer molecule requires a lower temperature to form a stable hybrid with a template. The primer sequence does not have to be completely complementary to the template, but is complementary enough to be hybridized with the template. The primer of the present invention may be chemically synthesized using a known method in the art, for example phosphoramidite solid support method. Also, the primer may be modified by methylation or capsulation according to a known method.

As used herein, the term “probe” refers to a nucleotide fragment of RNA or DNA which is composed of several bases to several hundreds of bases, and which can bind specifically to mRNA. Since the probes are labeled, they can be used for detecting the presence of certain mRNA. Probes may be prepared in the form of oligonucleotide probe, single stranded DNA probe, double stranded DNA probe, RNA probe, and the like.

A formulation measuring a PDK protein expression level may include an antibody specific for a protein encoded by the PDK gene.

As used herein, the term “antibody,” which is a known term in the art, refers to a specific protein molecule that is directed against an antigenic site. The antibody of the present invention is not specifically limited, and includes a polyclonal antibody, a monoclonal antibody, or part thereof with antigen-binding properties, and all immunoglobulin antibodies. Further, the antibody of the present invention includes a specific antibody, such as humanized antibody. The antibody used in the present invention includes functional fragments of the antibody molecule, as well as a complete form having two full-length light chains and two full-length heavy chains. Functional fragments of the antibody molecule mean fragments having at least an antigen-binding function, including Fab, F(ab′), F(ab′)2, Fv, and the like.

In an embodiment, the present invention provides a method for diagnosing chronic inflammatory pain, including measuring an expression level of mRNA of a PDK gene or a PDK protein from a biological sample.

A formulation measuring an mRNA expression level of the PDK gene includes an oligonucleotide, a primer, or a probe, which binds complementarily to mRNA of the PDK gene.

A formulation measuring a PDK protein expression level includes an antibody specific for a protein encoded by the PDK gene.

The biological sample of the present invention may include tissues, cells, whole blood, serum, plasma, salvia, sputum, cerebrospinal fluid, or urine, but is not limited thereto.

An mRNA expression level of the present invention may be measured by polymerase chain reaction (PCR), reverse transcription-polymerase chain reaction (RT-PCR), competitive reverse transcription-polymerase chain reaction (Competitive RT-PCR), real-time reverse transcription-polymerase chain reaction (Real-time RT-PCR), RNase protection assay (RPA), Northern blotting, or DNA chip, but is not limited thereto.

A PDK protein expression level of the present invention may be measured, using an antigen-antibody binding reaction with an antibody binding specifically to a protein encoded by the PDK gene, by Western blotting, enzyme linked immunosorbent assay (ELISA), radioimmunoassay (RIA), radioimmunodiffusion, Ouchterlony immune diffusion, rocket immunoelectrophoresis, tissue immunostaining, immunoprecipitation assay, complement fixation assay, fluorescence activated cell sorter (FACS), or protein chip, but is not limited thereto.

Using the above methods, the level of mRNA of the PDK gene or the protein encoded by the gene in a biological sample is compared with the mRNA or protein expression level in a normal group (control group). Thereby, when the mRNA or protein expression level of the PDK gene is significantly raised, it can be diagnosed to be chronic inflammatory pain.

In an embodiment, the present invention provides a kit for diagnosing chronic inflammatory pain containing the composition.

The kit for diagnosis of the present invention includes a type or more of different constituent compositions, a solution or a device, suitable for an assay. For example, the kit of the present invention may be a kit which includes genomic DNA derived from a sample to be analyzed, a primer set specific for the PDK gene of the present invention, a proper amount of DNA polymerase (for example, Taq-polymerase), deoxynucleotides (dNTP) mixture, PCR buffer solution, and water, to perform PCR. The PCR buffer solution may include KCl, Tris-HCl, and MgCl2. In addition, the kit of the present invention may further include a constituent component needed to perform the electrophoresis which is used to check the amplification of a PCR product.

Further, the kit of the present invention may be a kit which includes an essential element needed to perform RT-PCR. The RT-PCR kit may include PDK gene-specific primer pairs and also include a test tube or other appropriate containers, reaction buffer solution (with various pH and magnesium concentrations), dNTPs, an enzyme, such as Taq-polymerase and reverse transcriptase, DNase, RNase inhibitor, DEPC-water, sterilized water, and the like. Further, it may include a primer pair specific for a gene which serves as a quantitative control group.

Further, the kit of the present invention may be a kit which includes an essential element needed to perform DNA chip analysis. The DNA chip kit may include a substrate to which a PDK gene or cDNA corresponding to a fragment of the gene is attached as a probe. The substrate may also include a quantitative structural gene or cDNA corresponding to a fragment of the quantitative structural gene.

Further, the kit of the present invention may be a microarray kit which includes a substrate on which a PDK gene of the present invention is fixed.

The kit of the present invention may be a kit which includes an essential element needed to perform ELISA. The ELISA kit includes an antibody specific for a PDK protein, and a formulation measuring the level of the protein. The ELISA kit may include a reagent which can detect an antibody forming an antigen-antibody complex, for example, a labeled secondary antibody, chromophores, an enzyme (e.g., an enzyme conjugated to antibody), and a substance of the enzyme. Further, it may include an antibody specific for a quantitative control group protein. The antigen-antibody complex refers to a composite of a protein encoded by a PDK gene and an antibody specific therefor. The formation amount of the antigen-antibody complex may be quantitatively measured by the size of a signal of the detection label. The detection label may be selected from the group consisting of an enzyme, a fluorescent substance, a ligand, a luminescent substance, microparticles, a redox molecule, and radio isotope, but is not limited thereto.

In an embodiment, the present invention provides a method for screening an agent for treating chronic inflammatory pain, including treating a biological sample with a candidate substance for treating chronic inflammatory pain and measuring an expression level of mRNA of a PDK gene or a PDK protein.

The candidate substance is treated to the biological sample, and when the expression level of mRNA of the PDK gene or the PDK protein is reduced compared with the control group, it can be determined to be an agent for treating chronic inflammatory pain.

Hereinafter, examples are provided in order to help understand the present invention. However, the examples are for illustrative purposes and are not intended to limit the scope of the present invention.

### Example 1

### Construction of Chronic Inflammatory Pain Model

### 1-1. Preparation of Mice

All experiments were conducted in accordance with the animal care guidelines of the National Institutes of Health. Pdk2-KO, Pdk4-KO, and Pdk2/4-DKO mice were obtained from Dr. Nam Ho Jeoung (Catholic University of Daegu, Republic of Korea) and Dr. Robert A. Harris (Indiana University USA). Male Pdk2/4 wild-type (WT, Pdk2/4+/+), Pdk2 knockout (2-KO, Pdk2−/−), Pdk4 knockout (4-KO, Pdk4−/−) and Pdk2/4 double knockout (DKO, Pdk2/4−/−) mice aged 8 to 10 weeks were used. Specifically, to produce Pdk2−/−(homozygous Pdk2-KO mice) and Pdk4−/−(homozygous Pdk4-KO mice) C57BL/6J black mice, the method of (Jeoung et al., 2012) was used. Pdk2-KO mice were crossed with Pdk4-KO mice to produce Pdk2/4−/−(homozygous Pdk2/4-DKO mice). As a control group, wild-type C57BL/6J black mice (The Jackson Laboratory, Bar Harbor, Me., U.S.A.) were used. Their genotypes were confirmed by PCR of the genomic DNA. The mice were housed under a 12 hour light/dark cycle (lights on 07:00 to 19:00/lights off 19:00 to 07:00) at a constant temperature of 23±2° C. with food and water provided.

### 1-2. Chronic Inflammatory Pain Induced by Complete Freund's Adjuvant Injection

The mice prepared by Example 1-1 were anaesthetized with oxygen (3%) and isoflurane (2%) inhalation, and then injected with 30 μl of CFA (0.5 mg/ml) into the left hindpaws to induce chronic inflammatory pain. Mice serving as the control group were injected with 30 μl of saline solution (carrier) to left hindpaws.

### Example 2

### Confirmation of PDK Expression Pattern in Mice where Chronic Inflammatory Pain is Induced

In order to confirm the change in PDK expression upon the onset of chronic inflammatory pain, mice prepared by Example 1-2 were euthanized and perfused through the aorta with 0.1 M PBS to remove the blood of the deeply anesthetized mice. The lumbar spinal cord and hindpaw tissues were rapidly dissected. The spinal cord portions, corresponding to segments L4-L6, were divided into ipsilateral and contralateral sides. The hindpaw tissues samples were then immediately frozen in liquid nitrogen and instantly homogenized in Trizol reagent for total RNA isolation. Total RNA (2 μl) from each sample was reverse-transcribed into cDNA using a first strand cDNA synthesis kit (MBI Fermentas, Hanover, Germany). Thereafter, the reverse transcription-PCR (RT-PCR) was performed using an LDNA Engine Tetrad Peltier Thermal Cycler (MJ Research, Waltham, Mass.). To analyze PCR products, 10 μl of each PCR product was electrophoresed in 1% agarose gel and the expression was detected under UV light. GAPDH was used as an internal control group. The nucleotide sequences of the primers used in the RT-PCR are shown in Table 1.

TABLE 1

| | Nucleotide sequence |
|---|---|
| Pdk1 forward primer | 5′-GGC GGC TTT GTG |
| | ATT TGT AT-3′ |
| Pdk1 reverse primer | 5′-ATA TGG GCA ATC |
| | CGT AAC CA-3′ |
| Pdk2 forward primer | 5′-GTC TGC TGG ACA |
| | TCA TGG AAT-3′ |
| Pdk2 reverse primer | 5′-CAT AGG CGT CTT |
| | TCA CCA CAT-3′ |
| Pdk3 forward primer | 5′-AAG CAG ATC GAG |
| | CGC TAC TC-3′ |
| Pdk3 reverse primer | 5′-GGA AAG AAA TGC |
| | GGT TGG TA-3′ |
| Pdk4 forward primer | 5′-AGA GCC TGA TGG |
| | ATT TGG TG-3′ |
| Pdk4 reverse primer | 5′-TCG AAG AGC ATG |
| | TGG TGA AG-3′ |
| GAPDH forward primer | 5′-ACC ACA GTC CAT |
| | GCC ATC AC-3′ |
| GAPDH reverse primer | 5′-TCC ACC ACC CTG |
| | TTG CTG TA-3′ |

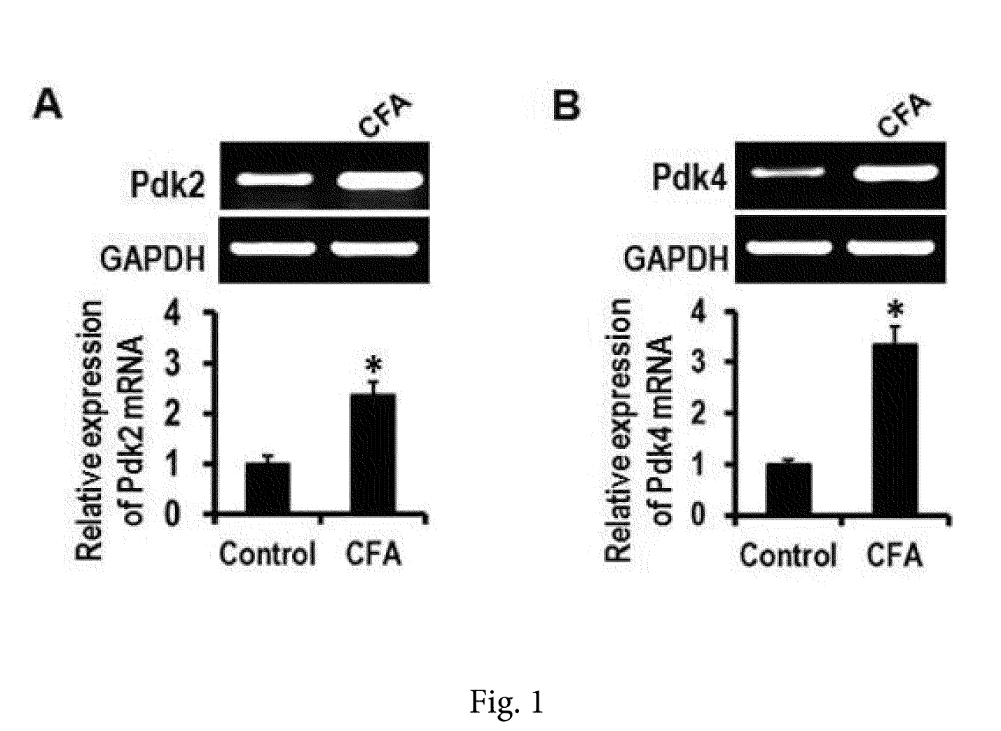

The mRNA expression pattern of PDK was confirmed through RT-PCT using the primers and the results are shown in FIG. 1.

As shown in FIGS. 1A and 1B, it was found that the mRNA expression of Pdk2 and Pdk4 increased in hindpaw tissues of mice where chronic inflammatory pain is induced by CFA. This demonstrates that chronic inflammatory pain can be diagnosed by measuring the PDK expression level.

### Example 3

### Confirmation of PDH Phosphorylation in Mice where Chronic Inflammatory Pain is Induced

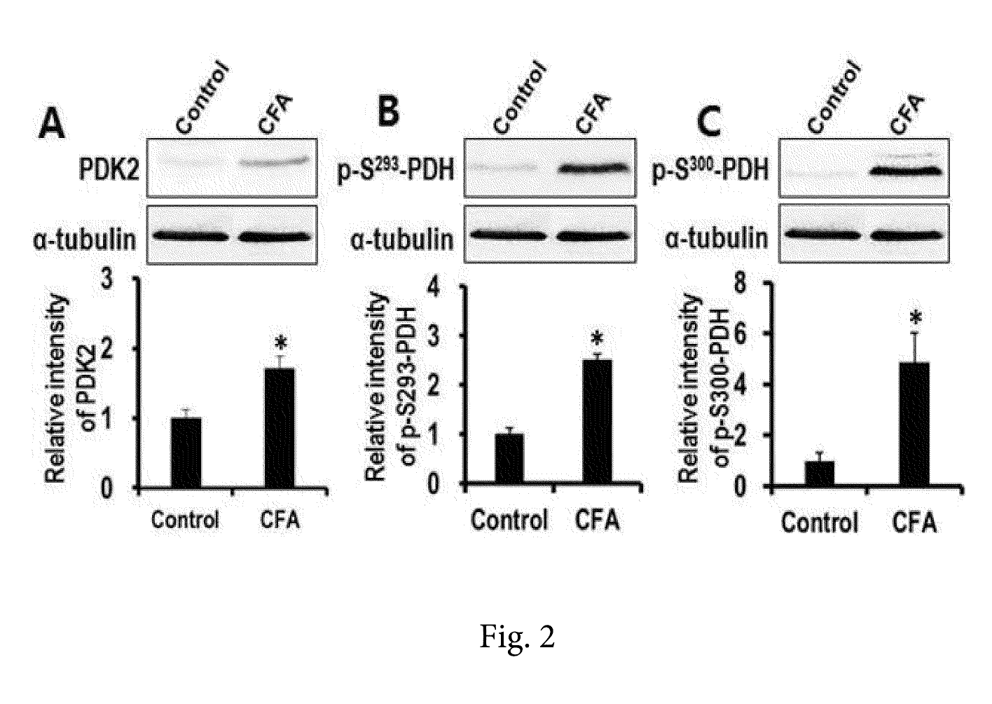

The PDK overexpression can be also confirmed indirectly by assessing the PDH phosphorylation. Thus, the PDH phosphorylation at the time of onset of chronic inflammatory pain was confirmed through Western blotting. Hindpaw tissues of similar weights at 3 days after CFA injection as in Example 1-2 were isolated from each mouse and washed in ice-cold PBS. Then, the tissue samples were placed in 300 μl of lysis buffer (150 mM sodium chloride, 1% Triton X-100, 1% sodium deoxycholate, 0.1% sodium dodecyl sulfate, 50 mM Tris-HCl (pH 7.5), 2 mM EDTA) (GenDEPOT, Barker, Tex.) containing protease inhibitor (1×) and phosphatase protease inhibitor cocktails (1×) (Thermo Scientific). The samples were individually homogenized, centrifuged with 13,400 g at 4° C. for 15 minutes. Protein was analyzed with a Bio-Rad Laboratories Protein Assay Kit using BSA as standard. Proteins (20 to 30 μg) from each sample were separated from 8 or 15% SDS-PAGE gels, and transferred to PVDF membrane (Bio-Rad, Hercules, Calif.) by the semi-dry electroblotting method. The membranes were blocked with 5% skim milk, reacted with primary antibodies (Rabbit monoclonal antibody; Acris) against PDK2, phospho-Ser²⁹³-PDHE1α (pyruvate dehydrogenase E1α) (Rabbit monoclonal antibody; Calbiochem, San Diego, Calif.), phospho-Ser³⁰⁰-PDHE1α (pyruvate dehydrogenase E1α) (Rabbit monoclonal antibody; Calbiochem, San Diego, Calif.), and α-tubulin (mouse monoclonal antibody; Sigma), and sequentially incubated with horseradish peroxidase-conjugated secondary antibodies (anti-rabbit and anti-mouse IgG antibody; Amersham Biosciences). Then, chemiluminescence was measured. The results are shown in FIG. 2.

As shown in FIG. 2, it was found that the PDK2 protein expression level and PDH phosphorylation (p-S²⁹³-PDH and p-S³⁰⁰-PDH) increase in hindpaw tissues of mice where chronic inflammatory pain is induced by CFA.

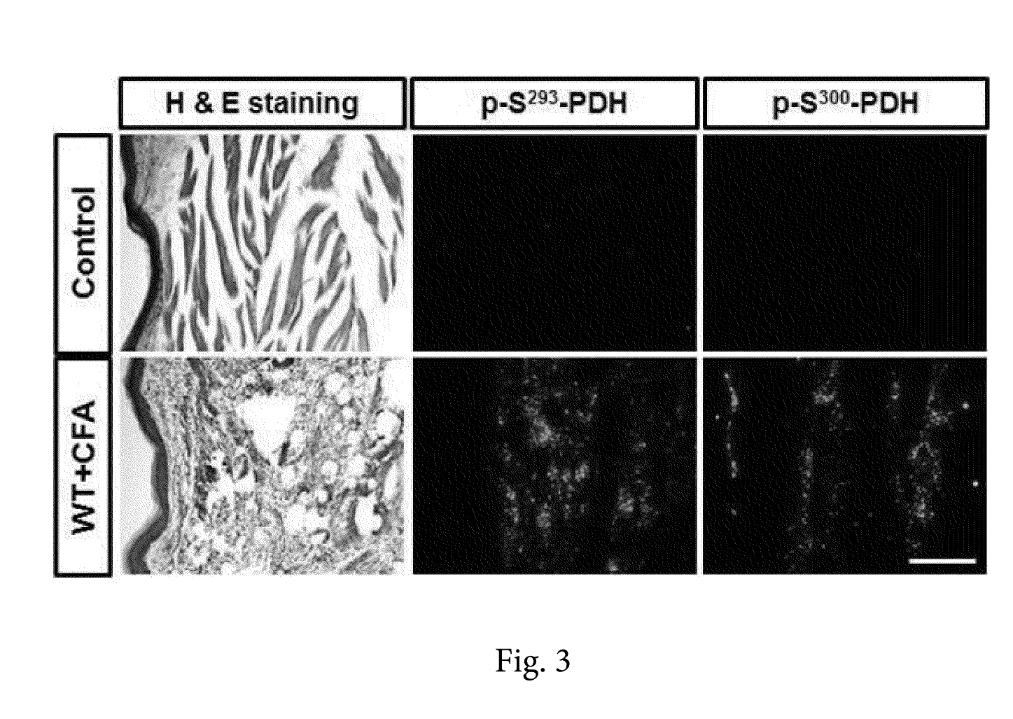

In order to verify the above experimental results, immunostaining was performed. First, deeply anesthetized mice by injecting CFA as in Example 1-2 were perfused through the aorta with 0.1 M PBS followed by 4% paraformaldehyde (PFA) fixative. Lumbar spinal cord segments (L4-L6) and hindpaw tissues were dissected out, post-fixed in the same PFA fixative overnight, and cryoprotected in 30% sucrose solution in 0.1 M PBS overnight at 4° C. A cryostat was used to prepare 10 μm sections of hindpaw tissues, which were fixed onto gelatin-coated slides, and 30 μm sections of spinal cord, which were placed in 0.1 M PBS. Sections were then blocked with 4% normal serum in 0.3% Triton X-100 for 90 minutes at room temperature. For immunofluorescence staining, the tissue sections were incubated with primary antibodies against phosphor-PDHE1α(pS²⁹³) (rabbit, 1:200; Calbiochem, San Diego, Calif.) and phosphor-PDHE1α (pS³⁰⁰) (rabbit, 1:200; Calbiochem, San Diego, Calif.) overnight at 4° C., and then incubated with FITC- or Cy3-conjugated secondary antibodies (1:200; Jackson ImmunoResearch, West Grove, Pa.). Slides were washed, coverslipped with Vectashield mounting medium (Vector Laboratories, Burlingame, Calif.), and visualized under a fluorescence microscope. The results are shown in FIG. 3.

As shown in FIG. 3, it was found that as the result of immunostaining hindpaw tissues where chronic inflammatory pain is induced by CFA injection, an immune response of the antibodies against p-S²⁹³-PDH and p-S³⁰⁰-PDH significantly increased, and that no change is observed in the control group administered with the carrier.

### Example 4

### Confirmation of Hindpaw Edema and Histological Features in Mice where Chronic Inflammatory Pain is Induced

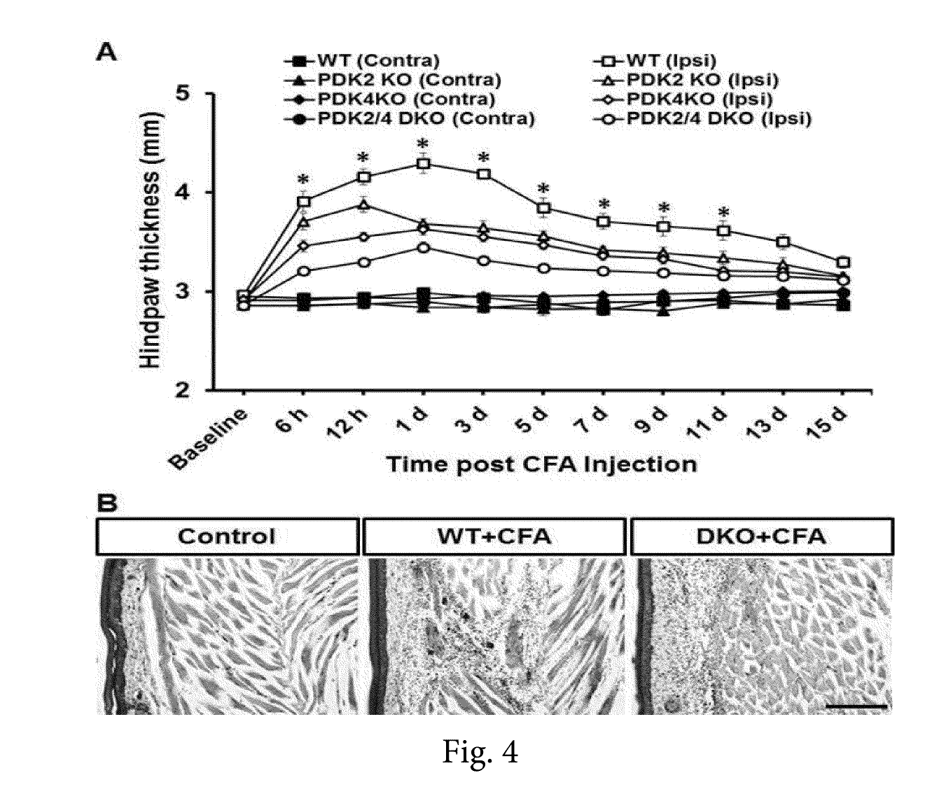

Hindpaw edema induced by CFA, which is a measure of the degree of inflammation, was measured through paw thicknesses. The dorsoventral thicknesses of the middle portions of the hindpaws were measured using a caliper (Jha et al., 2014). Hindpaw edema was measured up to 2 weeks after CFA injection, and was expressed in millimeters. The results are shown in FIG. 4A.

As shown in FIG. 4A, it was found that edema is diminished significantly lower in Pdk2 knockout mice, Pdk4 knockout mice, and Pdk2/4 knockout mice, than in WT mice after CFA injection.

Also, hindpaw tissues inducing chronic inflammatory pain by injecting CFA were immunestained using the same method as Example 3, and then analyzed with an optical microscope (Olympus B-50, Tokyo) to assess histological features. The results are shown in FIG. 4B.

As shown in FIG. 4B, it was observed that infiltration of inflammatory cells and damage to tissues occur due to the onset of chronic inflammatory pain in hindpaw tissues of mice injected with CFA, and it was found that the inflammatory response was attenuated in Pdk2/4 knockout mice.

### Example 5

### Assessment of Pain Behaviors in Mice where Chronic Inflammatory Pain is Induced

Thermal hyperalgesia and mechanical allodynia associated with inflammation were assessed by measuring the hindpaw withdrawal latencies or thresholds, respectively.

Thermal hyperalgesia was defined as a decrease in thermal paw withdrawal latencies (PWL) to a noxious thermal stimulus. PWLs were assessed using the Hargreaves' plantar test Analgesymeter (Ugo Basile, Comerio, Italy). Mice of Example 1-2 were acclimatized to the animal management room for 1 week, and placed in the laboratory used for the experiment for 1 hour 1 day before the experiment. Mice were acclimatized under a plastic chamber on a glass surface maintained at 30° C. The plantar surface of each hindpaw was then exposed to an electrically generated thermal stimulus (set at 5.0 A). The time required for a mouse to withdraw its paw from the stimulus (PWL) was automatically recorded. The intensity of the radiant heat was adjusted so that basal PWL was between 9 and 12 seconds, with a cut-off of 20 seconds to avoid any possible tissue damage.

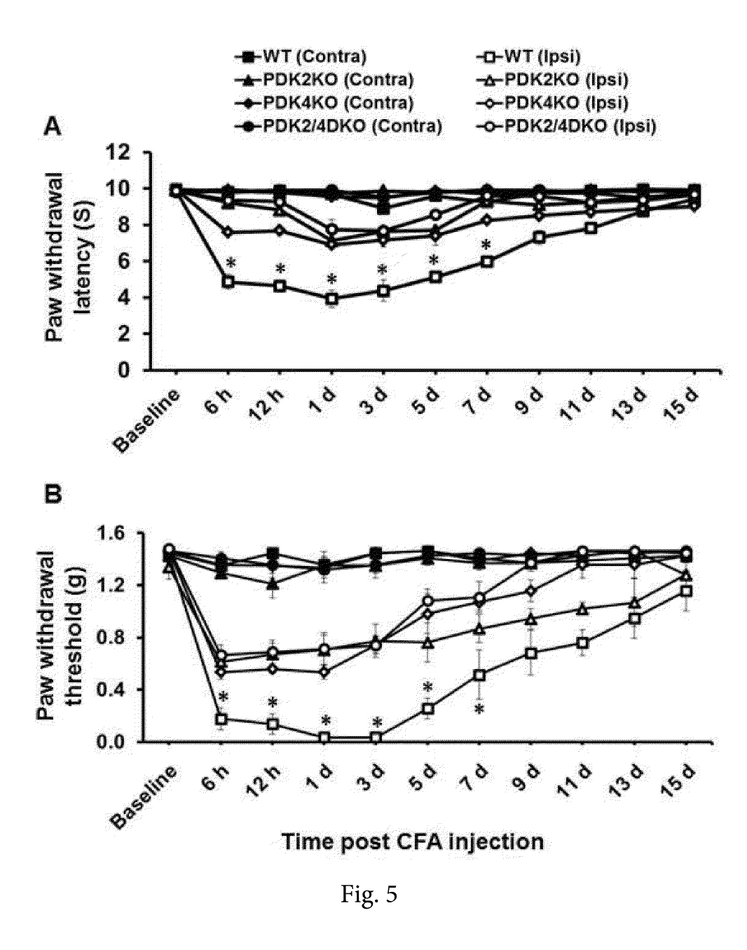

Mechanical allodynia was defined as a paw withdrawal threshold (PWT) required for avoiding pain, and the PWT was evaluated on two consecutive days before and after CFA injection using calibrated von Frey filaments (Bioseb™, Chaville, France) (Chaplan et al, 1994). Von Frey filaments were presented perpendicularly to the plantar surface, and held in position for approximately 5 seconds with enough force to cause a slight bend. A positive response was defined as abrupt paw withdrawal. The results are shown in FIG. 5.

As shown in FIG. 5, it was found that Pdk2 knockout mice, Pdk4 knockout mice and Pdk2/4 knockout mice at 6 hours to 7 days after CFA injection exhibited attenuated thermal hyperalgesia (FIG. 5A) and mechanical allodynia (FIG. 5B), when compared with WT mice.

### Example 6

### Measurement of Infiltration of Neutrophils, Macrophages and iNOS-Positive Immune Cells in Mice where Chronic Inflammatory Pain is Induced

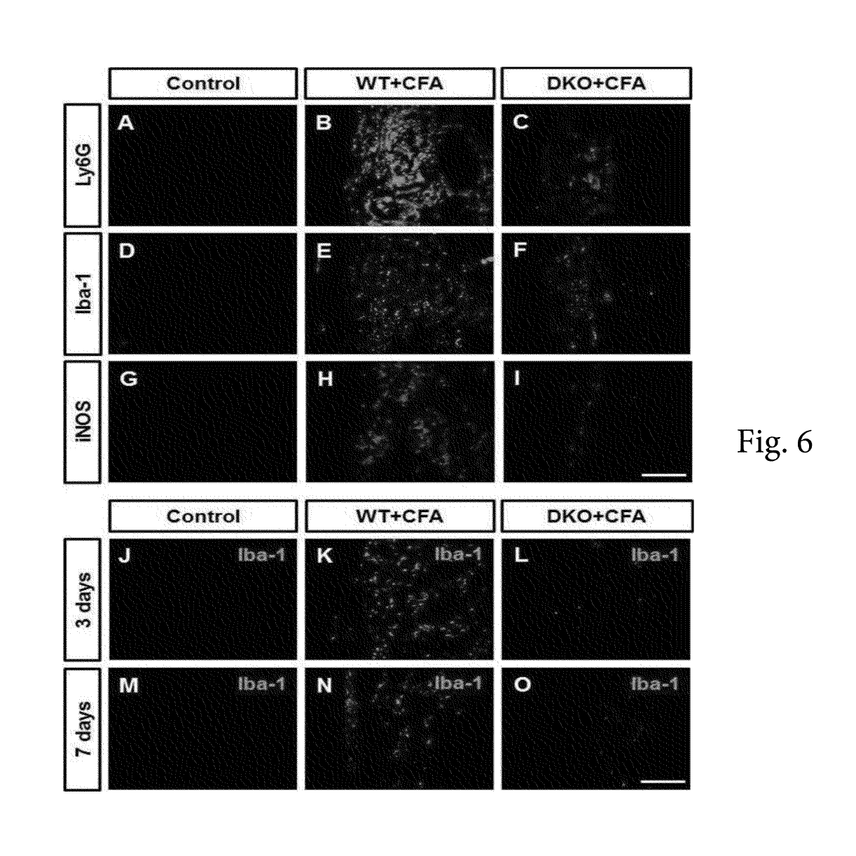

The infiltration of neutrophils, macrophages and iNOS-positive immune cells in hindpaw tissues of mice where chronic inflammatory pain is induced by injecting CFA as in Example 1-2 was immunestained using the same method as Example 3, and then analyzed with a fluorescence microscope. The results are shown in FIG. 6.

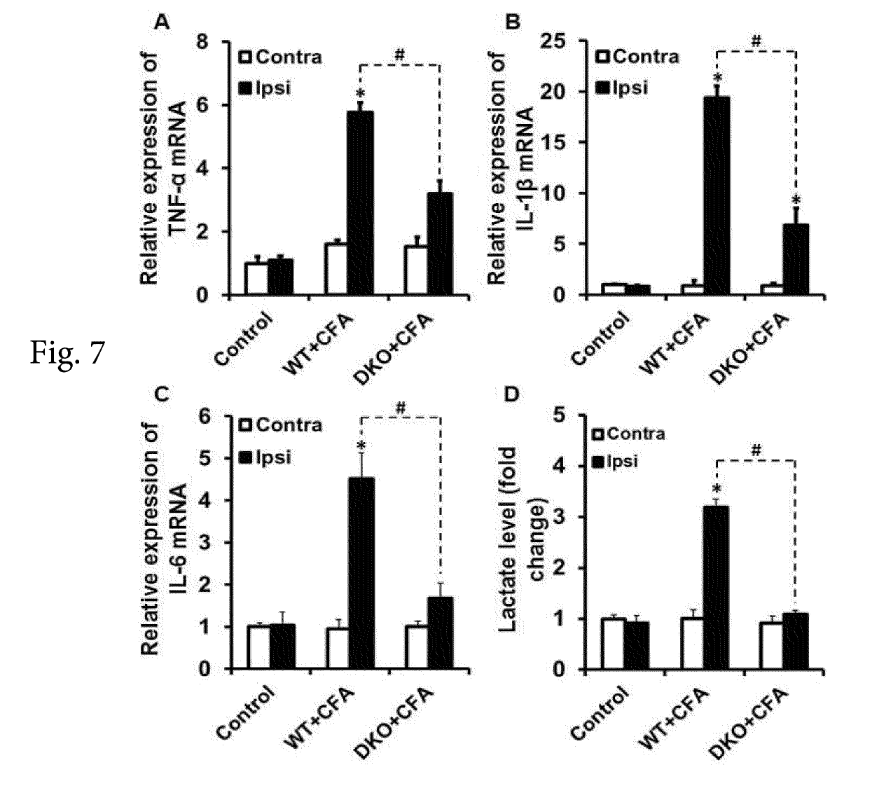

As shown in FIG. 6, the infiltration of neutrophils (Ly6G-positive cells) (FIGS. 6A, 6B, and 6C) and macrophages (Iba-1-positive cells)(FIGS. 6D and 6E) were attenuated at 1 day after CFA injection to hindpaw of Pdk2/4-deficient mice, when compared with WT mice, and the infiltration of iNOS-positive immune cells (FIGS. 6G, 6H, and 6I) were also attenuated at 3 days after CFA injection. Infiltration of neither neutrophil/macrophage nor iNOS-positive immune cells was observed in the hindpaw tissues of mice of the control group administered with carrier. Similarly, the persistent immunoreactivities revealing the infiltration of Iba-1-positive macrophages were detected at 3 days (FIGS. 6J, 6K, and 6L) and 7 days (FIGS. 6M, 6N, and 6O) after CFA injection. As a result, it was found that Pdk2/4-deficient mice exhibited attenuated infiltration compared with WT mice. It demonstrated that the infiltration of neutrophils, macrophages, and iNOS-positive immune cells were inhibited by Pdk2/4-deficiency.

### Example 7

### Measurement of Expression of TNF-α IL-1β and IL-6 and Lactate Level in Mice where Chronic Inflammatory Pain is Induced

3 days after CFA or carrier injection as in Example 1-2, mice were dissected, and the contralateral sides of the lumbar segment of spinal cords and hindpaws were observed. All the tissue samples were snap-frozen in liquid nitrogen. Upon the experiment, spinal cord and hindpaw tissues were homogenized respectively in 300 μl and 500 μl of lactate assay buffer solution (Lactate Colorimetric kit, Abcam) and centrifuged at 4° C. at 10,000 g for 4 minutes. Thereafter, the expression of TNF-α IL-1β and IL-6 and lactate level was measured, and the results are shown in FIG. 7.

As shown in FIGS. 7A, 7B, and 7C, it was found that the expression of TNF-α (FIG. 7A), IL-1β (FIG. 7B) and IL-6 (FIG. 7C) was significantly increased in the hindpaw tissues at 3 days after CFA central injection in WT mice, whereas the expression of pro-inflammatory cytokines was significantly attenuated in Pdk2/4 deficient mice.

Further, as shown in FIG. 7D, the lactate level at 3 days after CFA central injection in WT mice was increased by more than three folds compared to the control group administered with carrier. In Pdk2/4-deficient mice, the chronic inflammation induced by lactic acidosis in the hindpaw tissues having localized inflammation was diminished.

### Example 8

### Confirmation of Treatment Effect of PDK Inhibitor Administration on Chronic Inflammatory Pain

To confirm the treatment effect of PDK inhibitor on chronic inflammatory pain models, dichloroacetate (DCA), a PDK inhibitor, was administered to mice where chronic inflammatory pain is induced by CFA injection as in Example 1-2, and the edema and pain behaviors in mice were assessed according to the methods of Examples 4 and 5. To the control group, a carrier, instead of DCA, was administered.

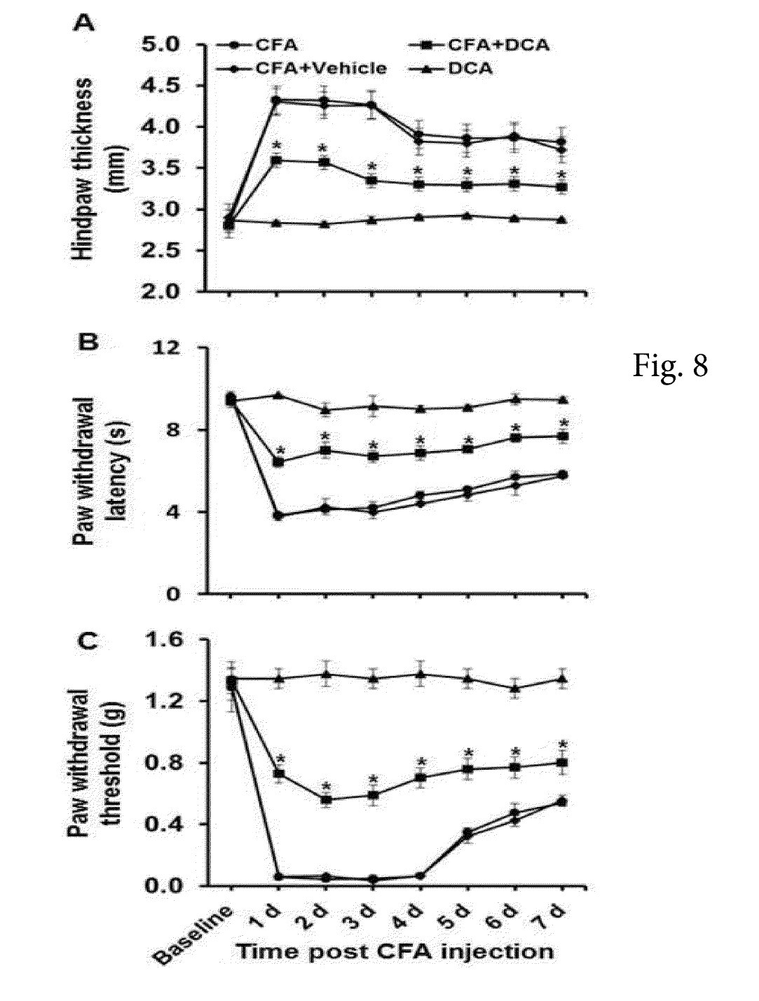

Peripheral administration of DCA (10 mg/kg body weight, 10 μl) or carrier (saline, 10 μl) was done by intraplantar injection at 48 hours after CFA injection. The results are shown in FIG. 8.

As shown in FIG. 8A, it was found that hindpaw edema increased by CFA injection in mice treated with DCA was significantly diminished, whereas no change was observed in the control group treated with carrier.

Further, as shown in FIGS. 8B and 8C, thermal hypersensitivities and mechanical allodynia were significantly reduced in mice treated with DCA, when compared with the control group.

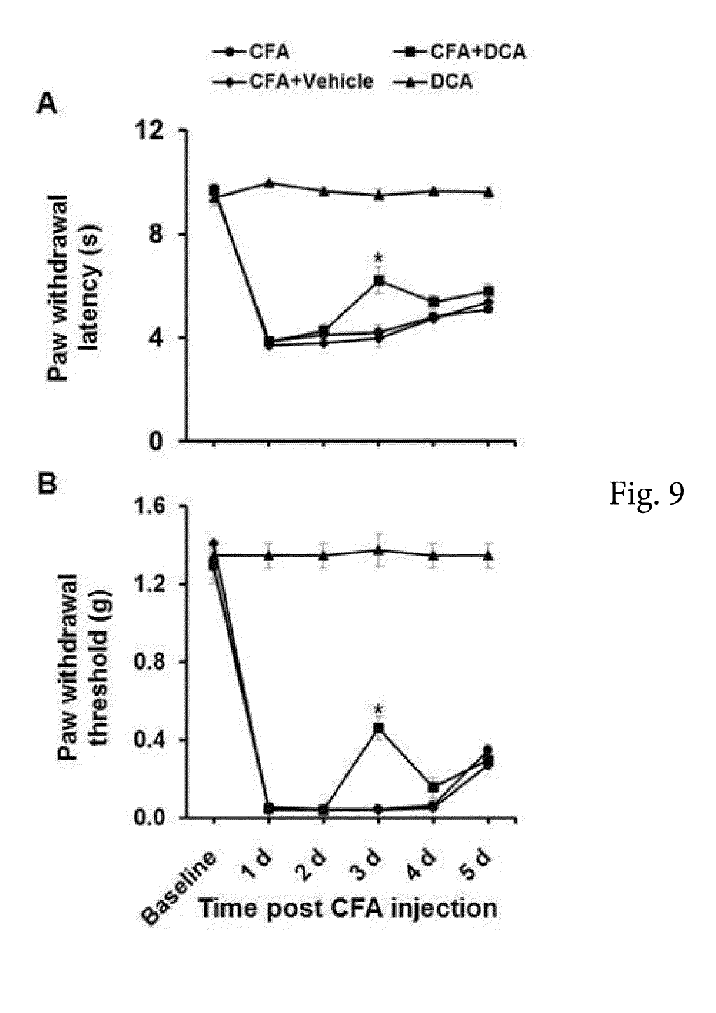

In the case of central administration, DCA (10 mg/kg body weight, 10 μl) or carrier (saline, 10 μl) was administered via intrathecal routes at 48 hours after CFA injection, and the results are shown in FIG. 9.

As shown in FIG. 9, it was demonstrated that thermal hypersensitivities (FIG. 9A) and mechanical allodynia (FIG. 9B) were significantly reduced in mice treated with DCA, when compared with the control group

From the above examples, it was confirmed that PDK expression increases when chronic inflammatory pain is induced, and thus it can be used in diagnosis of chronic inflammatory pain, that pain behaviors and hindpaw edema were diminished with the reduction of inflammatory response in PDK deficient mice or mice administered with PDK inhibitor, and thus that the PDK inhibitor of the present invention can be useful for treating chronic inflammatory pain.

Hereinafter, preparation examples of pharmaceutical compositions for treating chronic inflammatory pain and food compositions for alleviating chronic inflammatory pain, which include a PDK inhibitor of the present invention, will be described. However, the examples are for illustrative purposes and are not intended to limit the scope of the present invention.

### Preparation Example 1

### Preparation of Pharmaceutical Formulations

### 1. Preparation of Powders

| | |
|---|---|
| PDK inhibitor | 20 mg |
| lactose | 100 mg |
| talc | 10 mg |

The above ingredients were mixed and filled in a sealed pouch to prepare the formulation in the form of powders.

### 2. Preparation of Tablets

| | |
|---|---|
| PDK inhibitor | 10 mg |
| corn starch | 100 mg |
| lactose | 100 mg |
| magnesium stearate | 2 mg |

The above ingredients were mixed and tableted according to a general tableting method to prepare the formulation in the form of a tablet.

### 3. Preparation of Capsules

| | |
|---|---|
| PDK inhibitor | 10 mg |
| crystalline cellulose | 3 mg |
| lactose | 14.8 mg |
| magnesium stearate | 0.2 mg |

The above ingredients were mixed and filled into a gelatin capsule according to a general capsule preparation method to prepare the formulation in the form of a capsule.

### 4. Preparation of Injections

| | |
|---|---|
| PDK inhibitor | 10 mg |
| mannitol | 180 mg |
| sterile distilled water | 2974 mg |
| Na₂HPO₄2H₂O | 26 mg |

The formulation in the form of an injection was prepared with the above ingredients per ample (2 ml) according to a general injection preparation method.

### 5. Preparation of Liquids

| | | |
|---|---|---|
| PDK inhibitor | 20 | mg |
| isomerose | 10 | g |
| mannitol | 5 | g |

| | |
|---|---|
| purified water | adequate |

According to a general liquid preparation method, each of the above ingredients was added to purified water to be dissolved and added with an adequate amount of a lemon flavor. Then, the above ingredients were mixed together, and added with purified water to be a final volume of 100 ml. Thereafter, the mixture was filled into a brown bottle, and then sterilized to prepare the formulation in the form of a liquid.

### Preparation Example 2

### Preparation of Food Formulations

### 1. Preparation of Health Foods

| PDK inhibitor | 100 | mg |
|---|---|---|
| vitamin mixture | adequate | |
| vitamin A acetate | 70 | g |
| vitamin E | 1.0 | mg |
| vitamin B1 | 0.13 | mg |
| vitamin B2 | 0.15 | mg |
| vitamin B6 | 0.5 | mg |
| vitamin B12 | 0.2 | g |
| vitamin C | 10 | mg |
| biotin | 10 | g |
| nicotinic acid amide | 1.7 | mg |
| folic acid | 50 | g |
| calcium pantothenate | 0.5 | mg |
| mixture of minerals | adequate | |
| ferrous sulfate | 1.75 | mg |
| zinc oxide | 0.82 | mg |
| magnesium carbonate | 25.3 | mg |
| potassium phosphate, monobasic | 15 | mg |
| calcium phosphate, dibasic | 55 | mg |
| potassium citrate | 90 | mg |
| calcium carbonate | 100 | mg |
| magnesium chloride | 24.8 | mg |

The above ratio illustrates a preferred example of mixing the vitamins and the mixture of minerals relatively suitable for health foods. However, various modifications or changes in the mixing ratio may be possible. The above ingredients may be mixed according to a general health food preparation method to prepare granules and used for preparing health food compositions according to a general method.

### 2. Preparation of Health Beverages

| | | |
|---|---|---|
| PDK inhibitor | 100 | mg |
| vitamin C | 15 | g |
| vitamin E (powder) | 100 | g |
| ferrous lactate | 19.75 | g |
| zinc oxide | 3.5 | g |
| nicotinic acid amide | 3.5 | g |
| vitamin A | 0.2 | g |
| vitamin B1 | 0.25 | g |
| vitamin B2 | 0.3 | g |

| | |
|---|---|
| water | fixed amount |

According to a general health beverage preparation method, the above ingredients were mixed, stirred and heated at 85° C. for about 1 hour, and the prepared solution was filtered, filled into 2 L sterile containers, sealed, sterilized, kept under refrigeration, and then used for preparing a health beverage composition of the present invention.

The above ratio illustrates a preferred example of mixing ingredients relatively suitable for a beverage, but it may be arbitrarily modified according to regional and national preferences such as demand classes, demand nations, uses, and the like

1. A method for treating chronic inflammatory pain, the method comprising administering a pyruvate dehydrogenase kinase (PDK) inhibitor to a subject.

2. The method of claim 1, wherein the PDK is PDK-2 or PDK-4.

3. The method of claim 1, wherein the PDK inhibitor is a PDK activity inhibitor or a PDK expression inhibitor.

4. The method of claim 3, wherein the PDK inhibitor is a PDK activity inhibitor which is at least one member selected from the group consisting of: a compound, a peptide, a peptidomimetic, a substrate analog, an aptamer, and an antibody that binds specifically to a PDK protein.

5. The method of claim 3, wherein the PDK inhibitor is a PDK expression inhibitor which is at least one member selected from the group consisting of an antisense nucleotide, RNAi, siRNA, shRNA, and a ribozyme which binds complementarily to mRNA of a PDK gene.

6. The method of claim 1, wherein the PDK inhibitor is a pyruvate dehydrogenase complex (PDC) activator.

7. The method of claim 1, wherein the PDK inhibitor alleviates pain induced by complete Freund's adjuvant (CFA).

8. A method comprising: measuring an expression level of mRNA of a PDK gene or expression level of a PDK protein from a biological sample of a human suspected of having chronic inflammatory pain.

9. The method of claim 8, wherein an mRNA expression level of the PDK gene is measured employing an agent selected from the group consisting of: an oligonucleotide, a primer, and a probe which binds complementarily to mRNA of the PDK gene.

10. The method of claim 8, wherein a a PDK protein expression level is measured employing an antibody specific for the PDK protein.

11. The method of claim 8, wherein the biological sample is at least one member selected from the group consisting of: a tissue, a cell, whole blood, serum, plasma, salvia, sputum, cerebrospinal fluid, and urine.

12. The method of claim 8, wherein the mRNA expression level of the PDK gene is measured by at least one technique selected from the group consisting of: polymerase chain reaction (PCR), reverse transcription-polymerase chain reaction (RT-PCR), competitive reverse transcription-polymerase chain reaction (Competitive RT-PCR), real-time reverse transcription-polymerase chain reaction (Real-time RT-PCR), RNase protection assay (RPA), Northern blotting, and DNA chip.

13. The method of claim 8, wherein the PDK protein expression level is measured by at least one technique selected from the group consisting of: Western blotting, enzyme linked immunosorbent assay (ELISA), radioimmunoassay (RIA), radioimmunodiffusion, Ouchterlony immune diffusion, rocket immunoelectrophoresis, tissue immunostaining, immunoprecipitation assay, complement fixation assay, fluorescence activated cell sorter (FACS), and protein chip.

14. A method for screening an agent for treating chronic inflammatory pain, the method comprising: treating a biological sample with a candidate substance for treating chronic inflammatory pain and measuring an expression level of mRNA of a PDK gene or expression level of a PDK protein.


SEQUENCE LISTING (10 sequences)

SEQ ID NO: 1 - DNA, 20 bp, Artificial Sequence
  Synthetic DNA primer
ggcggctttgtgatttgtat

SEQ ID NO: 2 - DNA, 20 bp, Artificial Sequence
  Synthetic DNA primer
atatgggcaatccgtaacca

SEQ ID NO: 3 - DNA, 21 bp, Artificial Sequence
  Synthetic DNA primer
gtctgctggacatcatggaat

SEQ ID NO: 4 - DNA, 21 bp, Artificial Sequence
  Synthetic DNA primer
cataggcgtctttcaccacat

SEQ ID NO: 5 - DNA, 20 bp, Artificial Sequence
  Synthetic DNA primer
aagcagatcgagcgctactc

SEQ ID NO: 6 - DNA, 20 bp, Artificial Sequence
  Synthetic DNA primer
ggaaagaaatgcggttggta

SEQ ID NO: 7 - DNA, 20 bp, Artificial Sequence
  Synthetic DNA primer
agagcctgatggatttggtg

SEQ ID NO: 8 - DNA, 20 bp, Artificial Sequence
  Synthetic DNA primer
tcgaagagcatgtggtgaag

SEQ ID NO: 9 - DNA, 20 bp, Artificial Sequence
  Synthetic DNA primer
accacagtccatgccatcac

SEQ ID NO: 10 - DNA, 20 bp, Artificial Sequence
  Synthetic DNA primer
tccaccaccctgttgctgta